(19) United States
(12) Patent Application Publication
(10) Pub. No.: US 2015/0257698 A1
(43) Pub. Date: Sep. 17, 2015

(54) PATIENT FEEDBACK STIMULATION LOOP

(71) Applicant: ORIDION MEDICAL 1987 LTD., Jerusalem (IL)

(72) Inventors: Gregory Keith Spratt, Philadelphia, MO (US); Joshua Lewis Colman, Jerusalem (IL)

(73) Assignee: ORIDION MEDICAL 1987 LTD., Jerusalem (IL)

(21) Appl. No.: 14/216,090

(22) Filed: Mar. 17, 2014

(51) Int. Cl. A61B 5/00; A61B 5/0476; A61B 5/0205; A61B 5/0402

(52) U.S. Cl. CPC A61B 5/486; A61B 5/7264; A61B 5/4836; A61B 5/746; A61B 5/7246; A61B 5/0402; A61B 5/0476; A61B 5/0205; A61B 5/4821; A61B 5/7278; A61B 5/4812; A61B 5/742; A61B 5/741; A61B 5/7455; A61B 5/7275; A61B 5/0836

(57) ABSTRACT

Control logic, device and method including same configured to receive at least one measured medical parameter of a patient; compare the at least one measured medical parameter to a predetermined first threshold value and to a predetermined second threshold value; trigger stimulation of the patient when the at least one measured medical parameter crosses the first threshold value; and trigger an alarm when the at least one measured medical parameter crosses the second threshold value.

### TECHNICAL FIELD

The present disclosure relates generally to the field of patient stimulation and medical device alarms.

### BACKGROUND

Medical monitoring devices provide crucial data regarding a patient's medical condition. For example capnographs measure and provides values of the carbon dioxide (CO₂) concentration in exhaled breath, and as such may be used to characterize patient's ventilation functioning.

The medical devices are often configured to trigger an alarm alerting health care providers that a monitored parameter deviates from a threshold value. For example, a capnograph may set off an alarm when deviations or changes in the patient's CO₂ levels are detected.

### SUMMARY

Aspects of the disclosure, in some embodiments thereof, relate to patient stimulation feedback loops configured to stimulate a patient when a deviation in a monitored parameter is observed prior to activating traditional alarms alerting health care providers.

Frequent non-actionable alarms are a common complaint of caregivers. These alarms disrupt clinical workflow, are troubling to the patient and his or her surroundings, and may lead to alarm fatigue amongst the medical personnel. As a consequence thereof, true alerts may be overseen, as the alarm is ignored or even turned off, with a possibly tragic outcome.

The non-actionable alarms may not necessarily be false alarms. Rather, the sound of the alarm may increase the level of consciousness (LOC) of the patient and thereby stimulate the patient to breathe. Thus by the time the nurse or other medical personnel arrives the patient is breathing and the alarm is considered false.

The patient stimulation feedback loop disclosed herein, may be configured to stimulate the patient at some point prior to the activation of traditional alarms. The intent of the stimulation is to increase the LOC of the patient as low LOC is predictive of evolving respiratory compromise. The stimulation may also preemptively inform the patient, family and/or clinicians of indications of early respiratory compromise.

As a result, the patient stimulation feedback loop disclosed herein may significantly reduce alarm frequency by stimulating the patient prior to reaching an alarm threshold. This may on the one hand avoid disruption of clinician workflow while on the other hand increasing the clinician's confidence in the remaining alarms, consequently reducing the risk of them failing to notice a true alert.

Moreover, the patient and his surrounding may experience a calmer environment and reduced frustration and anxiety resulting from a feeling of being ignored and/or not attended to by the caregivers.

Certain embodiments of the present disclosure may include some, all, or none of the above advantages. One or more technical advantages may be readily apparent to those skilled in the art from the figures, descriptions and claims included herein. Moreover, while specific advantages have been enumerated above, various embodiments may include all, some or none of the enumerated advantages.

According to some embodiments, there is provided a control logic configured to: receive at least one measured medical parameter of a patient; compare the at least one measured medical parameter to a predetermined first threshold value and to a predetermined second threshold value; trigger stimulation of the patient when the at least one measured medical parameter crosses the first threshold value; and trigger an alarm when the at least one measured medical parameter crosses the second threshold value.

According to some embodiments, the at least one measured medical parameter may include a CO₂ related parameter, respiration rate (RR), an oxygen related parameter, heart rate (HR), an electrocardiogram (ECG), an encephalogram (EEG), blood pressure, spirometry, level of consciousness, level of sedation or any combination thereof. Each possibility is a separate embodiment.

According to some embodiments, the CO₂ related parameter may include a CO₂ waveform related parameter, an expired air CO₂ concentration, respiratory rate or any combination thereof. Each possibility is a separate embodiment.

According to some embodiments, the at least one measured medical parameter may include an algorithmically-derived index of multiple parameters. According to some embodiments, the algorithmically-derived index of multiple parameters is computed by:

(a) characterizing a first measured patient parameter based on a comparison of the first measured patient parameter against a first reference value;

(b) characterizing a second measured patient parameter based on a comparison of the second measured patient parameter against a second reference value; and

(c) computing the index value based on values associated with each of the characterized first and second measured patient parameters.

According to some embodiments, the first measured patient parameter may include a CO₂ related parameter, respiration rate (RR), an oxygen related parameter, heart rate (HR), an electrocardiogram (ECG), an encephalogram (EEG), blood pressure, spirometry, level of consciousness, level of sedation or any combination thereof. Each possibility is a separate embodiment.

According to some embodiments, the second measured patient parameter comprise CO₂ related parameter, respiration rate (RR), an O₂ related parameter, heart rate (HR), an electrocardiogram (ECG), an encephalogram (EEG), blood pressure, spirometry, level of consciousness, level of sedation or any combination thereof. Each possibility is a separate embodiment.

According to some embodiments, the at least one measured medical parameter is user selectable.

According to some embodiments, the first threshold value is tighter than the second threshold value. According to some embodiments, the first and second threshold values are user selectable.

According to some embodiments, the stimulation may include visual stimulation, audible stimulation, physical stimulation or combinations thereof. Each possibility is a separate embodiment.

According to some embodiments, the visual stimulation may include a flashing light. According to some embodiments, the audible stimulation may include a vocal instruction. According to some embodiments, the physical stimulation may include a vibration of a device attached to the patient.

According to some embodiments, the stimulation may trigger the patient to breathe. According to some embodiments, the stimulation may elevate the level of consciousness of the patient. According to some embodiments, the stimulation reduces the amount of non-actionable alarms.

According to some embodiments, the control logic is further configured to identify deterioration, lack of change or improvement of the at least one measured medical parameter within a predetermined period of time after the stimulation.

According to some embodiments, the control logic is further adapted to enhance the stimulation when no improvement in the at least one measured medical parameter is observed. According to some embodiments, the control logic is further adapted to trigger the alarm when no improvement in the at least one measured medical parameter is observed in response to the stimulation.

According to some embodiments, the control logic is further configured to store data including the measured medical parameter, changes in the measured medical parameter, type of stimulation applied, intensity of stimulation, number of stimulation cycles or combinations thereof.

According to some embodiments, there is provided a medical device comprising: at least one sensor configured to measure at least one medical parameter of a patient; and a control logic configured to: compare the at least one measured medical parameter to a predetermined first threshold value and to a predetermined second threshold value; trigger stimulation of the patient when the at least one measured medical parameter crosses the first threshold value; and trigger an alarm when the at least one measured medical parameter crosses the second threshold value.

According to some embodiments, there is provided a method for reducing non-actionable alarms, the method comprising: receiving at least one measured medical parameter of a patient; comparing the at least one measured medical parameter to a predetermined first threshold value and to a predetermined second threshold value; triggering stimulation of the patient when the at least one measured medical parameter crosses the first threshold value; and triggering an alarm when the at least one measured medical parameter crosses the second threshold value.

### BRIEF DESCRIPTION OF THE DRAWINGS

Some embodiments of the disclosure are described herein with reference to the accompanying figures. The description, together with the figures, makes apparent to a person having ordinary skill in the art how some embodiments of the disclosure may be practiced. The figures are for the purpose of illustrative discussion and no attempt is made to show structural details of an embodiment in more detail than is necessary for a fundamental understanding of the teachings of the disclosure. For the sake of clarity, some objects depicted in the figures are not to scale.

FIG. 1 schematically illustrates a medical device with a control logic according to some embodiments;

FIG. 2 is an illustrative flowchart of the operation of a control logic, according to some embodiments;

FIG. 3 is an illustrative flowchart of the operation of a control logic, according to some embodiments.

### DETAILED DESCRIPTION

In the following description, various aspects of the disclosure will be described. For the purpose of explanation, specific configurations and details are set forth in order to provide a thorough understanding of the different aspects of the disclosure. However, it will also be apparent to one skilled in the art that the disclosure may be practiced without specific details being presented herein. Furthermore, well-known features may be omitted or simplified in order not to obscure the disclosure.

The present disclosure relates generally to the field of patient stimulation and medical device alarms.

There is provided, according to some embodiments, a feedback stimulation loop comprising a control logic configured to receive at least one measured medical parameter of a patient; compare the at least one measured medical parameter to a predetermined first threshold value and to a predetermined second threshold value; trigger stimulation of the patient when the at least one measured medical parameter crosses the first threshold value; and trigger an alarm when the at least one measured medical parameter crosses the second threshold value.

As referred to herein, the terms “patient” and “subject” may interchangeably be used and may relate to a subject being monitored by any monitoring device for any physical-condition related parameter and/or health related parameter.

As used herein, the term “feedback stimulation loop” may refer to a stimulation provided to a patient in response to a deviation of a measured medical parameter from a predetermined threshold value.

As used herein, the term “stimulation” may refer to any stimulation provided to the patient capable of elevating the patient's level of consciousness (LOC).

As used therein the terms “level of consciousness” and “LOC” may interchangeably refer to a patient's responsiveness to stimuli from the environment. The LOC may range from alert to coma.

As used herein, the terms “alarm” and “traditional alarm” may interchangeable refer to an alarm configured to be triggered when a medical parameter crosses a predetermined threshold. The alarm is usually an audible alarm configured to alert the clinician. According to some embodiments, the clinician may be required to approach the patient in order to turn the alarm off.

According to some embodiments, the stimulation, optionally provided by a stimulation provider of the medical device, may increase the level of consciousness of the subject and/or reduces the amount of non-actionable alarms. According to some embodiments, the stimulation may trigger the patient to breathe.

According to some embodiments the stimulation may include any type of stimulation including visual stimulation, audible stimulation, physical stimulation or combinations thereof. According to some embodiments, the visual stimulation may be a flashing light. According to some embodiments, the audible stimulation may be a beep tone, a sound or music adapted to arouse the LOC of the patient. The audible stimulation may be different from the traditional alarm configured to alert the caregivers in order to facilitate the caregivers to distinguish between the traditional alarm and the patient stimulation. Additionally or alternatively, the audible stimulation comprises vocal instructions. For example, the audible stimulation may be a voice saying “take a deep breath”. Additionally or alternatively, the audible stimulation may be an instruction to reduce the dosage of patient-controlled analgesia (PCA). According to some embodiments, the physical stimulation may be a vibration of a device attached to the patient. Additionally or alternatively the physical stimulation may be a pressure applied to the patient for example by a pressure band or bracelet connected to the patient. It is understood by one of ordinary skill in the art that the described stimulations are illustrative only and that other stimulations capable of increasing the LOC of the patient are also applicable and as such fall within the present disclosure.

According to some embodiments, the stimulation type may be user selectable. For example the clinician can ask the patient as to which stimulation he prefers. Alternatively, the choice of stimulation may be based on a medical record of a patient.

It is further understood by the skilled in the art, that clinicians may provide instructions to the patient and/or his relatives as to how to respond to the stimulation provided. For example the patient may be instructed to take a deep breath, reduce PCA use or any other action suitable to the medical condition of the patient. Hence, the stimulation may, in additional to increasing the LOC of the patient, also assist in improving and/or halting deterioration of his or hers medical condition.

According to some embodiments, the feedback loop may be applied in post-operative settings, during sedation and/or anesthesia, in emergency settings, in intensive care units or any other suitable setting. Each possibility is a separate embodiment. As a non-limiting example, the feedback stimulation loop may be applied for capnographic monitoring of patients receiving sedation/analgesia, as the medications used may induce respiratory depression in addition to their targeted actions of relieving pain, anxiety, amnesia of disturbing procedures, etc.

According to some embodiments, the stimulation is provided when a change and/or deviation of at least one medical parameter from a predetermined threshold value is observed. As used herein, the term “at least one” with regards to medical parameters may refer to 1, 2, 3, 4, 5, 10 or more parameters. Each possibility is a separate embodiment.

According to some embodiments, the at least one measured medical parameter may include a CO₂-related parameter, respiration rate (RR), an oxygen-related parameter, heart rate (HR), an electrocardiogram (ECG), an encephalogram (EEG), blood pressure, spirometry, level of consciousness, level of sedation, or any combination thereof. According to some embodiments, the CO₂ related parameter may include a CO₂ waveform related parameter, an expired air CO₂ concentration, respiratory rate or any combination thereof. Each possibility is a separate embodiment.

According to some embodiments, the at least one measured medical parameter may be an algorithmically-derived index of multiple parameters. Hence, triggering of the stimulation may be induced by monitoring an algorithmically-derived index of multiple parameters rather than a single parameter.

According to some embodiments, the algorithmically-derived index of multiple parameters may be computed by:

(a) characterizing a first measured patient parameter based on a comparison of the first measured patient parameter against a first reference value;

(b) characterizing a second measured patient parameter based on a comparison of the second measured patient parameter against a second reference value; and

(c) computing the index value based on values associated with each of the characterized first and second measured patient parameters.

According to some embodiments, the at least one measured medical parameter is user selectable. It is understood by one of ordinary skill in the art that different medical parameters may be measured for different medical conditions. For example, following sedation a lower than normal respiration rate may be set to trigger the patient stimulation whereas a higher than normal respiration rate may be ignored.

According to some embodiment, the control logic may be configured to trigger stimulation when a first threshold value is crossed. According to some embodiments, the threshold value may be a predetermined deviation from a reference value. According to some embodiments, the term “reference value” may refer to a value, a range of values or a portion of a range of values representing a normal (healthy) condition. According to some embodiments, the term “threshold value” may refer to a value or a limit which when crossed initiates an action, such as, but not limited to, a patient stimulation or an alarm. The threshold value may be a fixed value or a value determined for example by the clinician based on the medical history of the patient monitored and/or the medical procedure the patient is undergoing or about to undergo. Each possibility is a separate embodiment.

According to some embodiments, the first threshold value may be tighter than the second threshold value. As used herein, the term “tighter” when referring to the threshold value may refer to a threshold value closer to the reference value. For example, the second threshold value (the alarm threshold value) may be set to trigger an alarm at high/low end-tidal carbon dioxide (EtCO₂) values of 60/20 mmHg, whereas the first threshold value (the patient stimulation threshold value) may be set at an EtCO₂ of 55/25 mmHg. For example, the alarm threshold value may be set to trigger an alarm at high/low respiration rate (RR) of 40/6 breaths per minute, whereas the patient stimulation threshold value may be set at 30/10 breaths per minute. For example, the alarm threshold value may be set to trigger an alarm at an oxygen saturation (SpO₂) below 85%, whereas the patient stimulation threshold value may be set at a SpO₂ below 90%. For example, the second threshold value, the alarm threshold value may be set to trigger an alarm at high/low pulse rate (PR) of 120/50 beats per minute, whereas the patient stimulation threshold value may be set at a PR of 110/55 beats per minute.

According to some embodiments, the first threshold value and the stimulation triggered when it is crossed may contribute to avoid deterioration in a medical condition of a patient. According to some embodiments, the first threshold value, and the stimulation triggered when it is crossed, may contribute to an early identification of respiratory compromise. According to some embodiments, the first threshold value, and the stimulation triggered when it is crossed, may contribute to an early identification of a respiratory disorder.

According to some embodiments, the first and second threshold values are user selectable. For example, the first threshold value may be tighter (closer to the reference value) for patient with low levels of consciousness. For example, the first threshold value may be tighter for patients known to encounter respiratory difficulties as compared to patient with no known medical history. For example, the first threshold value may be tighter for patients immediately after (or during a critical time period after) a medical procedure. For example, the first threshold value may be less tight for patients having an out of normal base line value (such as abnormally low pulse rates amongst athletes).

According to some embodiments, the control logic is configured to identify deterioration, lack of change or improvement of the at least one measured medical parameter within a predetermined period of time after the stimulation. The predetermined period of time after stimulation may be user-selectable. For example the predetermined period of time within which a response to the stimulation must be observed may include within 5 seconds, within 10 seconds, within 15 seconds, within 30 seconds or more. Each possibility is a separate embodiment.

According to some embodiments, if an improvement in the at least one measured medical parameter is observed, the second threshold value will not be crossed and the alarm configured to alert clinicians may not be triggered. According to some embodiments, if an improvement in the at least one measured medical parameter is observed, but the first threshold value remains to be crossed, the stimulation of the patient may be repeated. According to some embodiments, if an improvement in the at least one measured medical parameter is observed, but the first threshold value remains to be crossed, the stimulation of the patient may be enhanced (for example increasing the decibel level of the audible alarm). According to some embodiments, if an improvement in the at least one measured medical parameter is observed, but the first threshold value remains to be crossed, the stimulation of the patient may be changed (for example vibration may be replaced by a visual stimulation).

According to some embodiments, if a deterioration in the at least one measured medical parameter is observed, and the second threshold is crossed, the alarm configured to alert clinicians may be triggered. According to some embodiments, if a deterioration in the at least one measured medical parameter is observed, but the second threshold value is not yet crossed, the alarm configured to alert clinicians may not be triggered. According to some embodiments, if a deterioration in the at least one measured medical parameter is observed, but the second threshold value is not yet crossed, the stimulation of the patient may be repeated. According to some embodiments, if a deterioration in the at least one measured medical parameter is observed, but the second threshold value is not yet crossed, the stimulation of the patient may be enhanced (for example the duration and/or the intensity of the flashing light may be increased).

According to some embodiments, if no change in the at least one measured medical parameter is observed, the stimulation of the patient may be changed (for example vibration may be replaced by an audible stimulation). According to some embodiments, if no change in the at least one measured medical parameter is observed, the second threshold value will not be crossed and the alarm configured to alert clinicians may not be turned on. According to some embodiments, if no change in the at least one measured medical parameter is observed; the stimulation of the patient may be repeated. According to some embodiments, if no change in the at least one measured medical parameter is observed, the stimulation of the patient may be enhanced (for example vibration may be intensified). According to some embodiments, if no change in the at least one measured medical parameter is observed, the stimulation of the patient may be changed (for example vibration may be flashing light).

According to some embodiments, if no change in the at least one measured medical parameter is observed and/or if deterioration continues after at least two stimulation cycles, the alarm may be triggered even if the second threshold value has not been crossed. According to some embodiments, at least two stimulation cycles may refer to 2, 3, 4, 5 or more stimulation cycles. Each possibility is a separate embodiment. According to some embodiments, the at least two stimulation cycles may be identical (for example by repeating the stimulation). According to some embodiments, the at least two stimulation cycles may be of a different intensity and/or of a different duration. Each possibility is a separate embodiment. According to some embodiments, the at least two stimulation cycles may be of a different type. As a non-limiting example, the first stimulation may be physical and the second audible.

According to some embodiments, the control logic is configured to store data including, the measured medical parameter, changes in the measured medical parameter, type of stimulation applied, intensity of the stimulation applied, number of stimulation cycles provided or combinations thereof.

According to some embodiments, the data, or parts thereof, may be reported to the clinician. This may ensure that the clinician is aware of stimulation events. According to some embodiments, the reported data may further serve as a tool in the assessment of the patient's condition, for example once a traditional alarm is triggered.

According to some embodiments, there is provided a medical device including at least one sensor configured to measure at least one medical parameter of a patient; and a control logic. According to some embodiments, the control logic may be configured to compare the at least one measured medical parameter to a predetermined first threshold value and to a predetermined second threshold value; trigger stimulation of the patient when the at least one measured medical parameter crosses the first threshold value; and trigger an alarm when the at least one measured medical parameter crosses the second threshold value, as essentially described above.

According to some embodiments, the medical device may be a Capnograph, a CO₂ monitor, a respiration rate monitor, a heart rate monitor, a pulse monitor, an ECG monitor, an EEG monitor, a blood pressure monitor, an oxymeters, a spirometer or any combination thereof. Each possibility is a separate embodiment. According to some embodiment, the medical device is a capnograph. According to some embodiment, the medical device is a pulse oximeter.

According to some embodiments, the at least one sensor is a CO₂ sensor, a flow sensor, an infra-red (IR) sensor, a pulse oximetry sensor, a spirometer, a heart rate sensors, a pulse sensor, a respiration rate sensor a blood pressure sensors, an ECG, an EEG or combinations thereof. According to some embodiments, the term “at least one” when referring to a sensor may include 1, 2, 3, 4, 5 or more sensors. Each possibility is a separate embodiment.

According to some embodiments, there is provided a method for reducing non-actionable alarms. According to some embodiments the method may include receiving at least one measured medical parameter of a patient; comparing the at least one measured medical parameter to a predetermined first threshold value and to a predetermined second threshold value; triggering stimulation of the patient when the at least one measured medical parameter crosses the first threshold value; and triggering an alarm when the at least one measured medical parameter crosses the second threshold value.

According to some embodiments, the method may also include determining which medical parameters will be measured for example based on the medical record of the patient.

According to some embodiments, the at least one measured medical parameter may include a CO₂ related parameter, respiration rate (RR), an oxygen related parameter, heart rate (HR), an electrocardiogram (ECG), an encephalogram (EEG), blood pressure, spirometry, level of consciousness, level of sedation, or any combination thereof or any combination thereof. Each possibility is a separate embodiment. According to some embodiments, the CO₂ related parameter may include a CO₂ waveform related parameter, an expired air CO₂ concentration, respiratory rate or any combination thereof. Each possibility is a separate embodiment.

According to some embodiments, the at least one measured medical parameter may include an algorithmically-derived index of multiple parameters. Thus, according to some embodiments, the method may further include computing an index by:

(a) characterizing a first measured patient parameter based on a comparison of the first measured patient parameter against a first reference value;

(b) characterizing a second measured patient parameter based on a comparison of the second measured patient parameter against a second reference value; and

(c) computing the index value based on values associated with each of the characterized first and second measured patient parameters.

According to some embodiments, the method may further include setting/determining a first and/or second threshold value. According to some embodiments, setting the first threshold value may include determining the LOC of the patient.

According to some embodiments, the method may further include identifying a deterioration, lack of change or improvement in the at least one measured medical parameter within a predetermined period of time after the stimulation. According to some embodiments, the method may also include determining the predetermined period of time within which a change in the at least one measured parameter must be observed. According to some embodiments, determining the pre-determined period of time may include manually inserting the desired period of time, for example through a user interface. Alternatively, determining the pre-determined period may include choosing the desired period of time, for example from a scroll down menu. Suitable periods of time may include within 5 seconds, within 10 seconds, within 15 seconds, within 30 seconds or more. Each possibility is a separate embodiment.

According to some embodiments, the method further include selecting the type of stimulation provided to the patient, such as, but not limited to, an audible stimulation, a visible stimulation, a physical stimulation or combinations thereof. According to some embodiments, the method may also include determining the level or intensity of the stimulation provided. Additionally or alternatively the method may include determining the number of stimulation cycles which may be applied to the patient prior to triggering an alarm.

According to some embodiments, the method may further include storing data, such as, but not limited to, the measured medical parameter, changes in the measured medical parameter, type of stimulation applied, intensity of the stimulation applied, number of stimulation cycles provided or combinations thereof. According to some embodiments, the data, or parts thereof, may further be reported to the clinician.

Before explaining at least one embodiment in detail, it is to be understood that aspects of the embodiments are not necessarily limited in their application to the details of construction and the arrangement of the components and/or methods set forth herein. Some embodiments may be practiced or carried out in various ways. The phraseology and terminology employed herein are for descriptive purpose and should not be regarded as limiting.

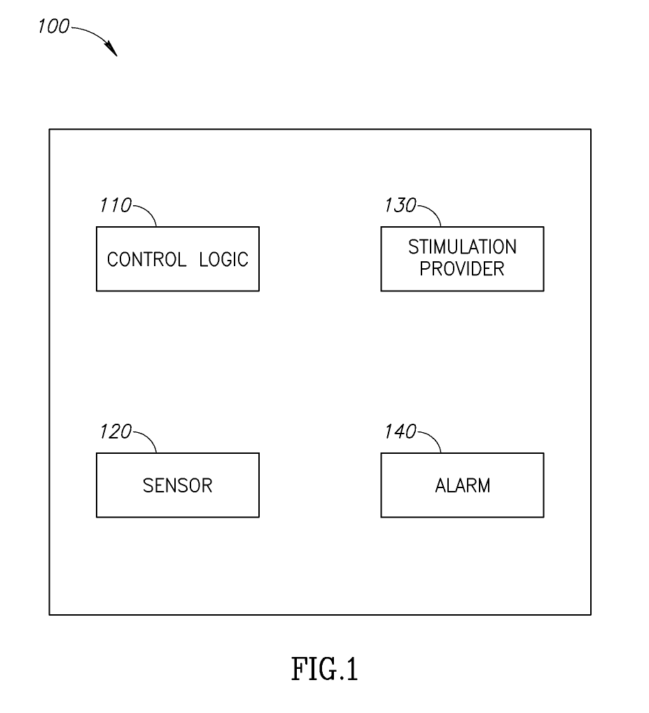

Reference is now made to FIG. 1, which schematically illustrates a medical device 100 with a control logic 110 according to some embodiments. Medical device 100 includes a sensor, here illustrated as a single sensor 120; however additional sensors or sensor assemblies are also applicable, as essentially described above. Control logic 110 of medical device 100 is configured to compare the medical parameter obtained from sensor 120 to a predetermined first threshold value and to a predetermined second threshold value. Medical device 100 further includes a stimulation provider 130 configured to be triggered when the measured medical parameter crosses the first threshold value. Medical device 100 also includes an alarm 140 configured to be triggered when the medical parameter crosses the second threshold value, as essentially described above.

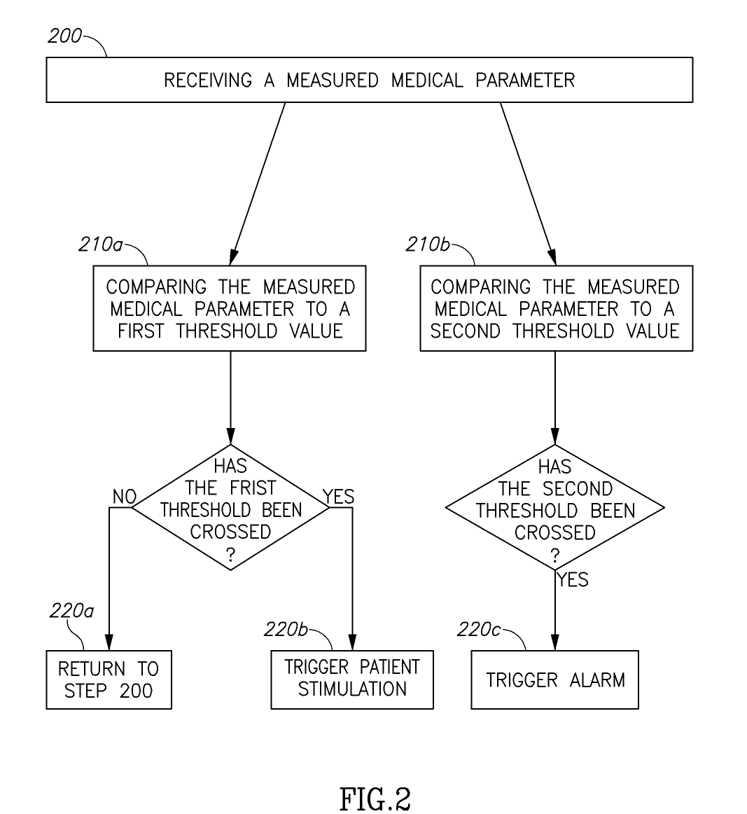

Reference is now made to FIG. 2 which is an illustrative flowchart of the operation of a control logic, according to some embodiments. At step 200, the control logic is configured to receive at least one medical parameter of a patient from a sensor(s) of a medical device. At step 210a and 210b, the medical parameter received is compared to a first and second threshold value, respectively. Has the first threshold value not been crossed, no action is triggered and the control logic is configured to return to step 200, as described in step 220a. Has the first threshold value been crossed, the control logic triggers stimulation of the patient, as described in step 220b. Has the second threshold value been crossed, the control logic triggers stimulation of an alarm configured to alert the medical personnel, as described in step 220c.

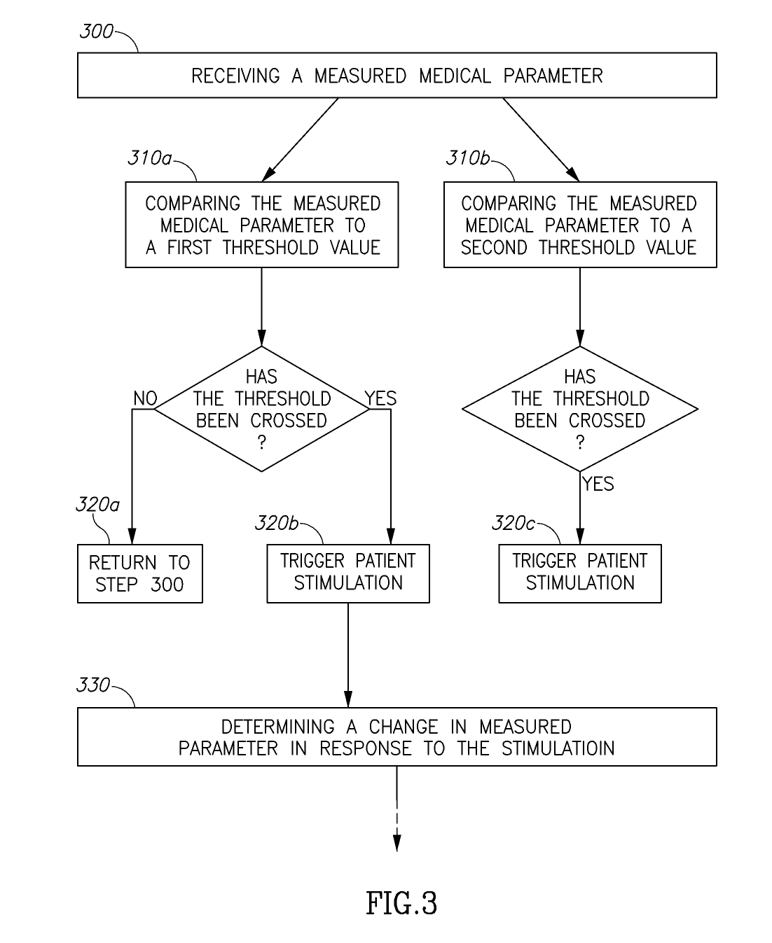

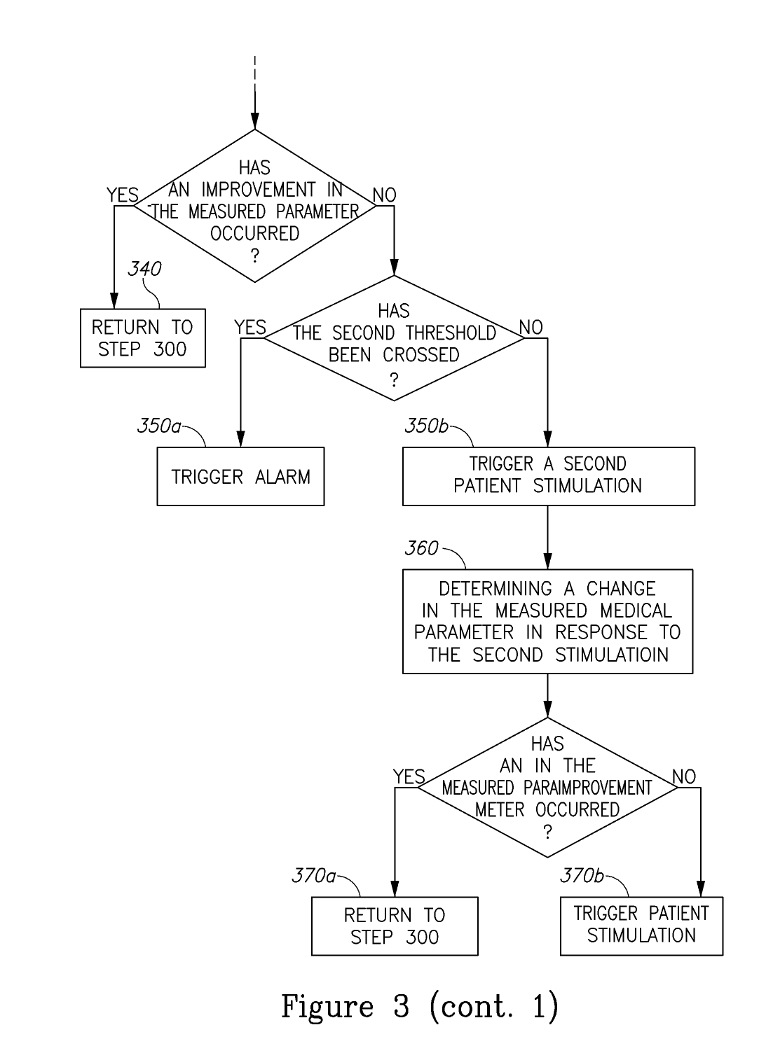

Reference is now made to FIG. 3 which is an illustrative flowchart of the operation of a control logic, according to some embodiments. At step 300, the control logic is configured to receive at least one medical parameter of a patient from a sensor(s) of a medical device. At step 310a and 310b, the medical parameter received is compared to a first and second threshold value, respectively. Has the first threshold value not been crossed, no action is triggered and the control logic is configured to return to step 300, as described in step 320a. Has the first threshold value been crossed, the control logic triggers stimulation of the patient, as described in step 320b. Has the second threshold value been crossed, the control logic triggers stimulation of an alarm configured to alert the medical personnel, as described in step 320b. If stimulation was provided, the control logic determines, in step 330, whether a change in the at least one measured medical parameter has occurred in response to the stimulation and whether the change is indicative of an improvement in the at least one measured medical parameter. Has an improvement been observed the control logic may return to step 300, as described in step 340. Has no improvement occurred, the control logic determines whether the second threshold has been crossed and if yes an alarm is triggered, as described in step 350a. If the second threshold has not been crossed, the control logic may trigger an additional stimulation, as described in step 350b, such as a repeated, enhanced and/or changed stimulation. In step 360 the control logic determines whether a change in the at least one measured medical parameter has occurred in response to the second stimulation and whether the change is indicative of an improvement in the at least one measured medical parameter. Has an improvement been observed, the control logic may return to step 300, as described in step 370a. Has no improvement occurred, the control logic triggers an alarm, as described in step 370b.

It is understood by one of ordinary skill in the art that variations may occur in the in the operation of the control logic. A non-limiting example of an optional variation may be adding additional stimulation cycles into the process. Variations may also occur in the decision taking steps of the process. For example, an improvement in the at least one medical parameter which still crosses the first threshold value may be set to trigger an alarm or to enhance stimulation. For example, an additional stimulation cycle may be added even if the at least one medical parameter returns to normal in order to maintain the elevated LOC of the patient.

The terminology used herein is for the purpose of describing particular embodiments only and is not intended to be limiting. As used herein, the singular forms “a”, “an” and “the” are intended to include the plural forms as well, unless the context clearly indicates otherwise. It will be further understood that the terms “comprises” or “comprising”, when used in this specification, specify the presence of stated features, integers, steps, operations, elements, or components, but do not preclude or rule out the presence or addition of one or more other features, integers, steps, operations, elements, components, or groups thereof.

While a number of exemplary aspects and embodiments have been discussed above, those of skill in the art will recognize certain modifications, additions and sub-combinations thereof. It is therefore intended that the following appended claims and claims hereafter introduced be interpreted to include all such modifications, additions and sub-combinations as are within their true spirit and scope.

What is claimed is:

1. A control logic configured to:
receive at least one measured medical parameter of a patient;
compare said at least one measured medical parameter to a predetermined first threshold value and to a predetermined second threshold value;
trigger stimulation of the patient when said at least one measured medical parameter crosses said first threshold value; and
trigger an alarm when said at least one measured medical parameter crosses said second threshold value.

2. The control logic of claim 1, wherein said stimulation triggers said patient to breathe.

3. The control logic of claim 1, wherein said at least one measured medical parameter comprises a CO₂ related parameter, respiration rate (RR), an oxygen related parameter, heart rate (HR), an electrocardiogram (ECG), an encephalogram (EEG), blood pressure, spirometry, level of consciousness, level of sedation or any combination thereof.

4. The control logic of claim 2, wherein said CO₂ related parameter comprises a CO₂ waveform related parameter, an expired air CO₂ concentration, respiratory rate or any combination thereof.

5. The control logic of claim 1, wherein said at least one measured medical parameter comprises an algorithmically-derived index of multiple parameters.

6. The control logic of claim 4, wherein said algorithmically-derived index of multiple parameters is computed by:
(a) characterizing a first measured patient parameter based on a comparison of the first measured patient parameter against a first reference value;
(b) characterizing a second measured patient parameter based on a comparison of the second measured patient parameter against a second reference value; and
(c) computing the index value based on values associated with each of the characterized first and second measured patient parameters.

7. The control logic of claim 5, wherein said first measured patient parameter comprise a CO₂ related parameter, respiration rate (RR), an oxygen related parameter, heart rate (HR), an electrocardiogram (ECG), an encephalogram (EEG), blood pressure, spirometry, level of consciousness, level of sedation or any combination thereof.

8. The control logic of claim 5, wherein said second measured patient parameter comprise CO₂ related parameter, respiration rate (RR), an O₂ related parameter, heart rate (HR), an electrocardiogram (ECG), an encephalogram (EEG), blood pressure, spirometry, level of consciousness, level of sedation or any combination thereof.

9. The control logic of claim 1, wherein said at least one measured medical parameter is user selectable.

10. The control logic of claim 1, wherein said first threshold value is tighter than said second threshold value.

11. The control logic of claim 1, wherein said first and second threshold values are user selectable.

12. The control logic of claim 1, wherein said stimulation comprises visual stimulation, audible stimulation, physical stimulation or combinations thereof.

13. The control logic of claim 7, wherein said visual stimulation comprises a flashing light; wherein said audible stimulation comprises a vocal instruction and wherein said physical stimulation comprises a vibration of a device attached to said patient.

14. The control logic of claim 1, wherein said stimulation increases the level of consciousness of the subject and/or reduces the amount of non-actionable alarms.

15. The control logic of claim 1, wherein said control logic is further configured to identify deterioration, lack of change or improvement of said at least one measured medical parameter within a predetermined period of time after said stimulation.

16. The control logic of claim 10, wherein said control logic is further adapted to enhance said stimulation when no improvement in said at least one measured medical parameter is observed.

17. The control logic of claim 10, wherein said control logic is further adapted to trigger said alarm when no improvement in said at least one measured medical parameter is observed in response to the stimulation.

18. The control logic of claim 10, wherein said control logic is further configured to store data including said measured medical parameter, changes in said measured medical parameter, type of stimulation applied, intensity of stimulation, number of stimulation cycles or combinations thereof.

19. A medical device comprising:
at least one sensor configured to measure at least one medical parameter of a patient; and
a control logic configured to:
compare the at least one measured medical parameter to a predetermined first threshold value and to a predetermined second threshold value;
trigger stimulation of the patient when said at least one measured medical parameter crosses said first threshold value; and
trigger an alarm when said at least one measured medical parameter crosses said second threshold value.

20. A method for reducing non-actionable alarms, the method comprising:
receiving at least one measured medical parameter of a patient;
comparing the at least one measured medical parameter to a predetermined first threshold value and to a predetermined second threshold value;
triggering stimulation of the patient when the at least one measured medical parameter crosses the first threshold value; and
triggering an alarm when the at least one measured medical parameter crosses the second threshold value.
